(19) United States
(12) Patent Application Publication
(10) Pub. No.: US 2025/0319229 A1
(43) Pub. Date: Oct. 16, 2025

(54) METHODS OF TREATING HARD TISSUE WITH BIOCOMPATIBLE MATERIAL

(71) Applicant: ROCHAL TECHNOLOGIES LLC, San Antonio, TX (US)

(72) Inventors: Nina BIONDA, Ithaca, NY (US); Rebecca E. McMAHON, San Antonio, TX (US); Ann Beal SALAMONE, Gainesville, FL (US)

(73) Assignee: ROCHAL TECHNOLOGIES LLC, San Antonio, TX (US)

(21) Appl. No.: 19/174,366

(22) Filed: Apr. 9, 2025

Related U.S. Application Data
(60) Provisional application No. 63/632,055, filed on Apr. 10, 2024.

(51) Int. Cl. A61L 26/00; C08L 79/00

(52) U.S. Cl. CPC A61L 26/0019; A61L 26/0066; C08L 79/00; A61L 2300/206; A61L 2300/406

(57) ABSTRACT

A method of treating a biological hard tissue that includes applying an antimicrobial composition to a biological hard tissue. The antimicrobial composition includes at least one polymeric biguanide; a vicinal diol component comprising at least one monoalkyl glycol and at least one monoalkyl glycerol, where a weight ratio of the at least one polymeric biguanide and the vicinal diol component ranges from 1:0.05 to 1:500, and a chelating agent, where a weight ratio of the at least one polymeric biguanide and the chelating agent ranges from 1:0.01 to 1:100. The biological hard tissue is selected from bone, cementum, dentin, enamel, keratin, or sclerenchyma.

### CROSS-REFERENCE TO RELATED APPLICATIONS

This application claims priority under 35 U.S.C. 119 (e) to U.S. Application No. 63/632,055, filed Apr. 10, 2024, the entirety of which is incorporated herein by reference.

### FIELD OF INVENTION

This invention relates generally to methods for the treatment of biological hard tissue. In particular, aspects of this invention relate to methods for preventing or treating infection while not injuring the biological hard tissue.

### BACKGROUND

Acute trauma and aging are key contributors to hard tissue injury. As these injuries may result in infection, methods for prevention and mitigation of infection are typically deployed.

Skeletal bones are injured by fracture, dislocation, and compression or they are structurally weakened due to aging or disease processes. Worldwide the age standardized incidence rate of bone fractures is 2296 fractures per 100,000 people with a lower incidence for women (1943 fractures) compared to men (2620 fractures) (GBD 2019). With aging, incidence rates increase to the point where 15,381 fractures occurred per 100,000 people aged 95 years or older which is thought to be primarily due to osteoporosis (GBD, 2019). Osteopenia and osteoporosis compromise maturation of osteoblast progenitor cells, proliferative osteoprogenitor cell activity, bone forming capacity of mature osteoblasts and osteoblastic response to chemical signaling (Hart, 2011). Hence, the importance of using an antimicrobial composition that selectively targets microbes but does not harm mammalian cell and tissue function.

Dental trauma affects approximately ⅓ of children and toddlers (primary teeth) and ⅕ of adolescents and adults (permanent teeth) (Lam, 2016). Dental hard tissue injury includes infraction, crown fractures, and root fractures. 26-76% of dental trauma results in loss of dental hard tissue of which bacterial infection is a significant contributing factor (Lam, 2016).

The CDC reports that 1.9% of operative procedures result in surgical site infections, including the range from superficial incisional infection to osteomyelitis, joint or bone infection, periprosthetic joint infection to organ/space surgical site infection (CDC, 2015).

Keratinized tissues, such as hair, finger and toenails, are readily infected by filamentous fungi (dermatophytes) as well as other yeasts and saprophytic molds. Onychomycosis is the cause of 40-50% of nail dystrophies and is difficult to eradicate. Long-term systemic treatments produce undesirable side effects, such as hepatotoxicity (Hainer, 2003).

Animals with a carapace (keratin) suffer from injury and disease that results in damage to the carapace. Epizoonotic shell disease (polymicrobial infection in the carapace) is of particular concern due decline in animal populations (Schaubeck, 2023). An antimicrobial composition which is effective in mitigating infection while not harming healthy tissue is desirable to assist in species viability.

Sclerenchyma is hard tissue found in plants (Simply Science). It is composed of dead sclerenchyma cells that have thick walls typically containing lignin and cellulose (60-80%). Sclerenchyma is in coconut husks, nuts, and the seed coat of legumes. 551 million tons of legumes were produced globally in 2021 with the US contributing 123 million tons (Dell'Olmo, 2023). Multiple bacterial, fungal, and viral microorganisms damage legumes and their seeds causing loss of crops and consequential hunger. A multi-species antimicrobial composition, such as the one used in this invention, can be applied to plants, such as legumes, to mitigate infection while not harming the plant.

Infection treatment of hard tissue may be systemic or topical. Systemic treatment is known to have systemic side effects to gut health and organ health (Mohsen, 2020). Topical antimicrobial or antibiotic treatments have been known to produce toxic side effects as well (Lineaweaver, 1985).

### SUMMARY OF THE INVENTION

The present invention provides methods for treatment of biological hard tissue to prevent, inhibit, or treat infections and which do not injure biological hard tissue. Biological hard tissue includes bone, cementum, dentin, enamel, keratin and sclerenchyma. An infection is a pathogenic invasion or growth of bacteria, fungi, yeast, viruses, protozoa, mycoplasma, or other microorganisms. Hard tissue injury can occur by various means, such as trauma, progressive disease, aging processes, foreign body presence, and infection. Infection may be present in each of these injury types.

In some embodiments, a method for treatment of biological hard tissue with an antimicrobial composition which is biocompatible with the biological hard tissue is provided. As used herein, “biocompatible” has its plain meaning and includes an ability to be in contact with a biological hard tissue without producing materially adverse effects. Adverse effects could include impairing surface properties, such as roughness, porosity, toxic impurity presence, surface energy, or cytotoxicity.

In some embodiments, a method for treatment of biological hard tissue with an antimicrobial composition comprising polymeric biguanides, vicinal diols and chelating agents is provided. As used herein, “antimicrobial composition” has its plain meaning and includes compositions that inhibit, prevent, or treat microbial infections.

In some embodiments, the antimicrobial composition described herein can be used to treat biological hard tissue to reduce and eliminate gram-positive bacteria, gram-negative bacteria, fungi, yeast, mold, protozoa, mycoplasma and viruses.

In some embodiments, the method disclosed herein comprises application of the antimicrobial composition as a solution, gel, powder, foam, spray, film, or dressing to the biological hard tissue.

In some embodiments, the method comprises application of the antimicrobial composition as a solution, gel, powder, foam, spray, dipping, film, or dressing to a device surface which may be in contact with a biological hard tissue.

It has been unexpectedly discovered that the antimicrobial composition comprising polymeric biguanides, vicinal diols, and chelators prevents microbial growth and/or infection, but does not injure biological hard tissue.

These and other objectives and advantages of the method described herein, some of which are specifically described and others that are not, will become apparent from the detailed description and claims that follow.

### BRIEF DESCRIPTION OF THE DRAWINGS

FIG. 1. Schematic of the experimental set up for the assessment of microbiocidal effectiveness using the bone explants.

FIG. 2. Picture of the bone bioreactor set up.

FIG. 3. Survival of S. aureus on bone explants.

FIG. 4. Metabolic activity of bone explants.

### DETAILED DESCRIPTION OF THE INVENTION

The present invention provides methods for treatment of biological hard tissues with an antimicrobial composition for infection mitigation which does not injure the biological hard tissue. The antimicrobial composition may be utilized for prophylactic infection prevention as well as for treatment of an existing infection.

Infection mitigation with the antimicrobial composition described herein can include treatment of hard tissue such as bone, including fractured bone and osteoderms; hard dental tissue (cementum, dentin and enamel); keratin (hair, nails, carapace, reptilian scales); and sclerenchyma (nut shells, seed coatings, legume seed coats).

The method of use for the antimicrobial composition varies depending on the hard tissue. In some embodiments, the hard tissue is a hard tissue of an animal or a hard tissue of a mammal. In some embodiments, the hard tissue is a plant hard tissue.

For instance, treatment of a mammalian bone fracture, vertebral injury, or for use during implant placement (e.g., knee replacement) could comprise irrigating the surgical site with a solution of the antimicrobial solution. Treatment of a dental hard tissue could comprise irrigation or use of an antimicrobial gel or a powder depending on the treatment site. The method of treatment for keratin could comprise spraying for application to larger or irregular surfaces. Dipping may be the preferred method for plant hard tissue (e.g., nuts, seeds, and legumes).

Examples of antimicrobial compositions useful in the methods described herein are disclosed in U.S. Pat. No. 8,829,053, the entirety of which is incorporated herein.

In some embodiments, the antimicrobial compositions comprise at least one antimicrobial polymeric biguanide and at least one antimicrobial vicinal diol, selected from a monoalkyl glycol, a monoalkyl glycerol, and a monoacyl glycerol. In some embodiments, the antimicrobial composition further comprises a metal ion chelating agent, a surfactant, or both. When the antimicrobial composition is aqueous, it can have a pH 4.5-7.0 and an osmolality of 10-320 mOsm/kg.

The method described herein provides for the use of specific antimicrobial compositions that unexpectedly exhibit exceptional antimicrobial activity, while not damaging the hard tissue to which the antimicrobial compositions are being applied. While not wishing to be bound by theory, it is possible that these antimicrobial compositions selectively target disruption of microbial biofilm and microbial structural components, such as cell walls, which results in death to microorganisms. Higher order animals and plants have different, and perhaps more complex, extracellular matrix compositions as well as cell structures which are not affected by these specific antimicrobial compositions.

In one aspect, a method of treating a biological hard tissue is provided. The method can include applying an antimicrobial composition to a biological hard tissue, where the antimicrobial composition comprises at least one polymeric biguanide; a vicinal diol component comprising at least one monoalkyl glycol and at least one monoalkyl glycerol, wherein a weight ratio of said at least one polymeric biguanide and said vicinal diol component ranges from 1:0.05 to 1:500, and a chelating agent, wherein a weight ratio of said at least one polymeric biguanide and said chelating agent ranges from 1:0.05 to 1:100.

In some embodiments, the weight ratio of said at least one polymeric biguanide and said vicinal diol component is at least 1:0.10, or at least 1:0.25, or at least 1:0.5, or at least 1:0.75, or at least 1:1, or at least 1:2. In some embodiments, the ratio of said at least one polymeric biguanide and said vicinal diol component is not more than 1:400, or not more than 1:300, or not more than 1:200, or not more than 1:100, or not more than 1:75, or not more than 1:50, or not more than 1:25, or not more than 1:20, or not more than 1:15, or not more than 1:10, or not more than 1:8.

In some embodiments, the weight ratio of said at least one polymeric biguanide and said vicinal diol component ranges from 1:1 to 1:10 or from 1:1.5 to 1:8, or from 1:2 to 1:6, or any combination of these upper and lower ranges.

In some embodiments, the weight ratio of said at least one polymeric biguanide and said chelating agent is at least 1:0.1, or at least 1:0.2, or at least 1:0.4, or at least 1:0.5, or at least 1:0.6, or at least 1:0.75. In some embodiments, the weight ratio of said at least one polymeric biguanide and said chelating agent is not more than 1:75, or not more than 1:50, or not more than 1:25, or not more than 1:20, or not more than 1:15, or not more than 1:10, or not more than 1:8.

In some embodiments, the weight ratio of said at least one polymeric biguanide and said chelating agent ranges from 1:0.25 to 1:5, or from 1:0.4 to 1:3, or from 1:0.6 to 1:2, or any combination of these upper and lower ranges.

In some embodiments, the antimicrobial composition is biocompatible with biological hard tissue.

In some embodiments, the application step occurs during a surgical procedure. In some embodiments, the application step occurs during a surgical procedure where the biological hard tissue is accessed through an incision. In some embodiments, the biological hard tissue is bone.

For example, in some embodiments, the surgical procedure can include removing a portion of bone and replacing the portion of bone with an implant device. In some embodiments, the method can include removing a portion of bone and applying the antimicrobial composition to an exposed portion of the bone prior to attaching the implant device to the exposed portion of the bone. In some embodiments, the surgical procedure can be selected from procedures including, but not limited to, a knee replacement, a hip replacement, a fracture repair, and spinal surgery.

In some embodiments, the application step occurs via irrigation, surgical dressing, gel coating, powder coating of the bone. In some embodiments, the application step occurs via irrigation, surgical dressing, gel coating, powder coating of an implant device. In some embodiments, the application step occurs via soaking, irrigation, gel coating, powder deposition, or covering with a dressing.

In some embodiments, the biological hard tissue is selected from bone, cementum, dentin, enamel, keratin, or sclerenchyma. In some embodiments, the biological hard tissue is at least one of cementum, dentin, or enamel.

In some embodiments, the antimicrobial composition is a solution. In some embodiments, the solution is an aqueous solution. In some such embodiments, a pH of the aqueous solution can be from 4.5 to 7.0, or from 5.0 to 6.5.

In other embodiments, the solution can be an organic solution. For example, the antimicrobial composition can be an alcohol-based solution. For example, the composition can be ethanol-based or isopropanol-based.

In some embodiments, the chelating agent is present at a concentration of from 0.01 weight % to 1 weight %, wherein the percentage is based on the weight of the antimicrobial composition. In some embodiments, the chelating agent is present in an amount of at least 0.02 weight %, or at least 0.04 weight %, or at least 0.06 weight %, or at least 0.08 weight %, or at least 0.09 weight %. In some embodiments, the chelating agent is present in an amount of not more than 0.8 weight %, or not more than 0.6 weight %, or not more than 0.5 weight %, or not more than 0.4 weight %, or not more than 0.25 weight %.

In some embodiments, the at least one polymeric biguanide is present in an amount ranging from 0.01 to 1.5 weight %. In some embodiments, the at least one polymeric biguanide is present in an amount of at least 0.02 weight %, or at least 0.04 weight %, or at least 0.05 weight %, or at least 0.06 weight %, or at least 0.08 weight %, or at least 0.09 weight %. In some embodiments, the at least one polymeric biguanide is present in an amount of not more than 1.25 weight %, or not more than 1.0 weight %, or not more than 0.75 weight %, or not more than 0.5 weight %, or not more than 0.25 weight %.

In some embodiments, the vicinal diol component is present in an amount ranging from 0.05 to 6.0 weight %, based on the total weight of the antimicrobial composition. In some embodiments, the vicinal diol component is present in an amount of at least 0.1 weight %, or at least 0.15 weight %, or at least 0.2 weight %, or at least 0.25 weight %, or at least 0.3 weight %. In some embodiments, the vicinal diol component is present in an amount of not more than 5 weight %, or not more than 2.5 weight %, or not more than 1.5 weight %, or not more than 1 weight %, or not more than 0.75 weight %,

In some embodiments, the biological hard tissue is keratin. In some such embodiments, the application step occurs via soaking, irrigation, gel coating, powder deposition or covering with a dressing.

In some embodiments, the biological hard tissue is sclerenchyma. In some such embodiments, the application step occurs via spraying, soaking, gel coating or powder deposition.

In some embodiments, the antimicrobial composition is present in a concentrated composition (e.g., a powder). In some such embodiments, the active ingredients of the antimicrobial composition will be present at a higher concentration than in the liquid formulations (e.g., solutions) described herein. Thus, while the ratios of the active ingredients will not change, their compositions will.

For example, in some embodiments, the chelating agent is present in an amount of at least 5 weight %, or at least 7.5 weight %, or at least 10 weight %, or at least 12.5 weight %, or at least 15 weight %. In some embodiments, the chelating agent is present in an amount of not more than 30 weight %, or not more than 25 weight %, or not more than 20 weight %.

In some embodiments, the at least one polymeric biguanide is present in an amount of at least 5 weight %, or at least 7.5 weight %, or at least 10 weight %, or at least 12.5 weight %, or at least 15 weight %. In some embodiments, the at least one polymeric biguanide is present in an amount of not more than 30 weight %, or not more than 25 weight %, or not more than 20 weight %.

In some embodiments, the vicinal diol component is present in an amount of at least 30 weight %, or at least 40 weight %, or at least 50 weight %, or at least 55 weight %, or at least 60 weight %. In some embodiments, the at least one vicinal diol component is present in an amount of not more than 90 weight %, or not more than 80 weight %, or not more than 75 weight %, or not more than 70 weight %.

In some embodiments, the method includes mixing a powder version of the antimicrobial composition with a solvent. In some such embodiments, the solvent is an aqueous solvent.

In some embodiments, the vicinal diol component comprises at least one of the following:
(i) a monoalkyl glycol having the following structure,

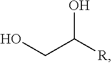

wherein R═C₃-C₁₈, branched or unbranched alkyl group or alkylene group; or
(ii) a monoalkyl glycerol having the following structure,

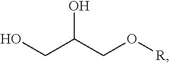

wherein R═C₃-C₁₈ branched or unbranched alkyl group or alkylene group.

In some embodiments, the polymeric biguanide comprises poly(hexamethylene biguanide) and its salts.

In some embodiments, the antimicrobial composition further comprises a bis(biguanide) at a concentration of from 10 ppm to 350 ppm. In some embodiments, the bis(biguanide) is present in an amount of at least 10 ppm, or at least 12 ppm, or at least 15 ppm, or at least 20 ppm, or at least 25 ppm, or at least 55 ppm, or at least 75 ppm. In some embodiments, the bis(biguanide) is present in an amount of not more than 350 ppm, or not more than 300 ppm, or not more than 250 ppm, or not more than 200 ppm, or not more than 175 ppm, or not more than 150 ppm.

In some embodiments, the polymeric biguanide is poly(hexamethylene biguanide). Generally, the hexamethylene biguanide polymer is also referred to as poly(hexamethylene biguanide) (PHMB), poly(hexamethylene bisbiguanide) (PHMB), poly(hexamethylene guanide) (PHMB), poly(aminopropyl biguanide) (PAPB), poly[aminopropyl bis(biguanide)] (PAPB), polyhexanide and poly(iminoimidocarbonyl)iminohexamethylene hydrochloride. PHMB is the preferred abbreviation for this biocidal polymer.

In some embodiments, the antimicrobial composition comprises at least one polymeric biguanide or polymeric bis(biguanide). Optionally, at least one low molecular weight bis(biguanide) can be added as an additional antimicrobial agent. The combination of multiple antimicrobial biguanides may enhance efficacy against the number and type of pathogenic microbial species.

In some embodiments, the antimicrobial composition comprises biocidal polymeric biguanides at a concentration of 0.05 wt % (500 ppm) to 1 weight % (10,000 ppm), more preferably between 0.075 wt % (750 ppm) to 0.5 wt % (5,000 ppm), and most preferably between 0.1 wt % (1,000 ppm) to 0.15 wt % (1,500 ppm). To this can be added bis(biguanides), such as alexidine and its salts and chlorhexidine and its salts. In some embodiments, the concentration of biocidal bis(biguanides) can range from 10 ppm (0.001 wt %) to 350 ppm (0.035 wt %). In some embodiments, the antimicrobial composition is an aqueous composition.

In some embodiments, a monoalkyl glycol is present and can include at least one of caprylyl glycol (also known as 1,2-dihydroxyoctane, 1,2-octanediol, and 1,2-octylene glycol), propylene glycol, hexylene glycol, 2-methyl-2,4-pentanediol, 1,3-butylene glycol, triethylene glycol, and glycol bis(hydroxyethyl) ether. In some embodiments, the monoalkyl glycol is a combination of caprylyl glycol (1,2-dihydroxyoctane) and propylene glycol. In some embodiments, the monoalkyl glycol is a combination of caprylyl glycol, a component of Sensiva® SC 10 with glycerol 1-(2-ethylhexyl) ether (2-ethylhexylglycerin).

In some embodiments, a monoalkyl glycerol is present and can include at least one of 1-O-heptylglycerol, 1-O-octylglycerol, 1-O-nonylglycerol, 1-O-decylglycerol, 1-O-undecylglycerol, 1-O-dodecylglycerol, 1-O-tridecylglycerol, 1-O-tetradecylglycerol, 1-O-pentadecylglycerol, 1-O-hexadecylglycerol (chimyl alcohol), 1-O-heptadecylglycerol, 1-O-octadecylglycerol (batyl alcohol), 1-O-octadec-9-enyl glycerol (selachyl alcohol), glycerol 1-(2-ethylhexyl) ether (also known as octoxyglycerin, 2-ethylhexyl glycerin, 3-(2-ethylhexyloxy) propane-1,2-diol, and SensivaR SC 50), 2-ethylhexyl diglycol ether, 2-ethylhexyl oligoglycol ethers, glycerol 1-heptyl ether, glycerol 1-octyl ether, glycerol 1-decyl ether, and glycerol 1-dodecyl ether, glycerol 1-tridecyl ether, glycerol 1-tetradecyl ether, glycerol 1-pentadecyl ether, glycerol 1-hexadecyl ether and glycerol 1-octadecyl ether. In some embodiments, the monoalkyl glycerol comprises glycerol 1-(2-ethylhexyl) ether, (Sensiva® SC 50) and 1-O-dodecylglycerol. In some embodiments, the monoalkyl glycerol is glycerol 1-(2-ethylhexyl) ether.

In some embodiments, monoacyl glycerols are present and can include at least one of 1-O-decanoylglycerol (monocaprin), 1-O-undecanoylglycerol, 1-O-undecenoylglycerol, 1-O-dodecanoylglycerol (monolaurin, also called glycerol monolaurate and Lauricidin®), 1-O-tridecanoylglycerol, 1-O-tetradecanoylglycerol (monomyristin), 1-O-pentadecanoylglycerol, 1-O-hexadecanoylglycerol, 1-O-heptadecanoylglycerol, and 1-O-octanoylglycerol (monocaprylin). In some embodiments, the monoacyl glycerols can include at least one of 1-O-decanoylglycerol, 1-O-dodecanoylglycerol, 1-O-tetradecanoylglycerol, or 1-O-octanoylglycerol. In some embodiments, the monoacyl glycerols can include 1-O-dodecanoylglycerol. In some embodiments, monoacyl glycerols substituted in the 1-O-position are more preferred than those substituted in the 2-O-position or disubstituted in the 1-O and 2-0 positions. In some embodiments, the monoacyl glycerols are hydrophobic.

In some embodiments, SensivaR SC 10 Multifunctional Cosmetic Ingredient, which includes both 1,2-octanediol (a monoalkyl glycol) and 2-ethylhexyl glycerin (glycerol 1-(2-ethylhexyl) ether) (a monoalkyl glycerol), is a vicinal diol composition for use in the antimicrobial compositions described herein.

In some embodiments, the antimicrobial composition comprises a biocidal hydrophobic monoalkyl glycol, a hydrophobic monoalkyl glycerol, and a hydrophobic monoacyl glycerol. In some embodiments, the combination of the biocidal hydrophobic monoalkyl glycol, the hydrophobic monoalkyl glycerol, and the hydrophobic monoacyl glycerol is present at a combined concentration of from 0.05 wt % (500 ppm) to 4 wt % (4,000 ppm), or between 0.1 wt % (1,000 ppm) to 1 wt % (10,000 ppm), or between 0.3 wt % (4,000 ppm) to 0.6 wt % (6,000 ppm).

In some embodiments, the antimicrobial composition is applied directly to the biological hard tissue. In other embodiments, the antimicrobial composition is applied indirectly to the biological hard tissue. For example, in some embodiments, the antimicrobial composition is applied to a dressing or to a medical device that will be placed in contact with the hard tissue.

When the antimicrobial composition is applied to a dressing or medical device in either the hydrated or dried form, the solution can contain polymeric biguanides at a concentration of 0.05 wt % (500 ppm) to 1 weight % (10,000 ppm), or from 0.075 wt % (750 ppm) to 0.75 wt % (7,500 ppm), or from 0.1 wt % (1,000 ppm) to 0.5 wt % (5,000 ppm), and antimicrobial monoalkyl glycols, antimicrobial monoalkyl glycerols, and monoacyl glycerols at a concentration of from 0.05 wt % (500 ppm) to 4 wt % (4,000 ppm), or from 0.1 wt % (1,000 ppm) to 1 wt % (10,000 ppm), or from 0.3 wt % (3,000 ppm) to 0.6 wt % (6,000 ppm).

It has been determined that chelating agents enhance the susceptibility of bacteria and other organisms to the biocidal effects of the antimicrobial agent, thus rendering an antimicrobial composition containing a chelating agent more effective in combating infection. Additionally, chelating agents deactivate matrix metalloproteases (MMPs), enzymes that can impede tissue formation and healing by breaking down collagen. MMPs are often found at elevated levels in injured tissue. Chelating agents bind to zinc ions, which are necessary for MMP activity, disrupting the MMP, causing deactivation, and thus facilitating healing.

The chelating agent is selected from any compound that is able to sequester monovalent or polyvalent metal ions, such as sodium, lithium, rubidium, cesium, calcium, magnesium, barium, cerium, cobalt, copper, iron, manganese, nickel, strontium or zinc, and is pharmaceutically or veterinary acceptable. The outermost surface of bacterial cells universally carries a net negative charge, which is usually stabilized by divalent cations such as Mg+2 and Ca+2. This is associated with the teichoic acid and polysaccharide elements of Gram-positive bacteria, the lipopolysaccharide of Gram-negative bacteria, and the cytoplasmic membrane itself. Thus, the chelating agent aids in destabilizing microorganisms.

Suitable chelating agents comprise, but are not limited to, aminocarboxylic acids, ethylenediaminetetraacetic acid (EDTA), 2-hydroxy-1,2,3-propanetricarboxylic acid (citric acid), nitrilotriacetic acid, nitrilotripropionic acid, diethylenetriaminepentaacetic acid, 2-hydroxyethylethylenediaminetriacetic acid, 1,6-diaminohexamethylenetetraacetic acid, 1,2-diaminocyclohexanetetraacetic acid, O,O′-bis(2-aminoethyl)ethyleneglycoltetraacetic acid, 1,3-diaminopropanetetraacetic acid, N,N′-bis(2-hydroxybenzyl)ethylenediamine-N,N′-diacetic acid, ethylenediamine-N,N′-diacetic acid, ethylenediamine-N,N′-dipropionic acid, triethylenetetraaminehexaacetic acid, ethylenediamine-N,N′-bis(methylenephosphonic acid), iminodiacetic acid, N,N-bis(2-hydroxyethyl)glycine, 1,3-diamino-2-hydroxypropanetetraacetic acid, 1,2-diaminopropanetetraacetic acid, ethylenediaminetetrakis(methylenephosphonic acid), N-(2-hydroxyethyl)iminodiacetic acid and biphosphonates such as editronate, and salts thereof. Suitable chelating agents include for example but are not limited to hydroxyalkylphosphonates, specifically the tetrasodium salt of 1-hydroxyethylidene-1,1-diphosphonic acid, also referred to as tetrasodium etidronate.

In some embodiments, the chelating agents are mixed salts of EDTA such as disodium, trisodium, tetrasodium, dipotassium, tripotassium, tetrapotasssium, lithium, dilithium, ammonium, diammonium, triammonium, tetraammonium, calcium and calcium-disodium, more preferably disodium, trisodium or tetrasodium salts of EDTA. In particular, in some embodiments, the chelating agents include disodium EDTA, trisodium EDTA, or both.

In some embodiments, the antimicrobial composition is an aqueous composition comprising chelating agents at a concentration of from 0.01 wt % (100 ppm) to 1 wt % (10,000 ppm), or from 0.025 wt % (250 ppm) to 0.5 wt % (5,000 ppm), or from 0.05 wt % (500 ppm) to 0.2 wt % (2,000 ppm). The chelating agent enhances biocidal activity by removing multivalent metal ions from microbial surfaces as well as potentially facilitating healing by deactivating matrix metalloproteases to enhance tissue regeneration.

The subject antimicrobial compositions can include one or more additional surfactants to effect surface cleaning. Suitable surfactants include for example but are not limited to cationic, anionic, nonionic, amphoteric and ampholytic surfactants. In some embodiments, the surfactants can be nonionic and amphoteric surfactants, with the nonionic surfactants most preferred. In some embodiments, the surfactants can have an HLB (hydrophilic-lipophilic balance) value of 18-30 in order to maintain the biocidal activity of the antimicrobial composition, while facilitating a non-cytotoxic solution.

Suitable nonionic surfactants include the ethylene oxide/propylene oxide block copolymers of poloxamers, reverse poloxamers, poloxamines, and reverse poloxamines. Poloxamers and poloxamines are preferred, and poloxamers are most preferred. Poloxamers and poloxamines are available from BASF Corp. under the trade names of Pluronic® and Tetronic®.

Suitable Pluronic surfactants comprise but are not limited to Pluronic F38 having a HLB of 31 and average molecular weight (AMW) of 4,700, Pluronic F68 having a HLB of 29 and AMW of 8,400, Pluronic 68LF having a HLB of 26 and AMW or 7,700, Pluronic F77 having a HLB of 25 and AMW of 6,600, Pluronic F87 having a HLB of 24 and AMW of 7,700, Pluronic F88 having a HLB of 28 and AMW or 11,400, Pluronic F98 having a HLB of 28 and AMW of 13,000, Pluronic F108 having a HLB of 27 and AMW of 14,600, Pluronic F127 having a HLB of 18-23 and AMW of 12,600, and Pluronic L35 having a HLB of 19 and AMW of 1,900. In some embodiments, the pluronic surfactant is Pluronic F127 (also known as Poloxamer 407).

Another class of surfactant is that of the diamino block copolymers of ethylene oxide and propylene oxide sold by BASF Corp. under the trade name Tetronic®. In some embodiments, the surfactant of this type is Tetronic 1107 (also known as Poloxamine 1107).

In addition to the above, other surfactants may be added, such as for example polyethylene glycol esters of fatty acids, e.g., coconut, polysorbate, polyoxyethylene or polyoxypropylene ethers of higher alkanes (C12-C18), polysorbate 20 available under the trademark Tween 20, polyoxyethylene (23) lauryl ether available under the trademark Brij 35, polyoxyethyene (40) stearate available under the trademark Myrj 52, and polyoxyethylene (25) propylene glycol stearate available under the trademark Atlas G 2612. Other neutral surfactants include nonylphenol ethoxylates such as nonylphenol ethoxylates, Triton X-100, Brij surfactants of polyoxyethylene vegetable-based fatty ethers, Tween 80, decyl glucoside, and lauryl glucoside.

Amphoteric surfactants suitable for use in solutions according to the present invention include materials of the type offered commercially under the trademark Miranol. Another useful class of amphoteric surfactants is exemplified by cocoamidopropyl betaine, commercially available from various sources.

One or more hydrophobic monoalkyl or monoalkylene alcohol can also be added to the antimicrobial composition to enhance biocidal activity. The monoalkyl alcohol can include a single hydroxyl group, i.e., is not a polyol. The monoalkyl alcohol can be solubilized by a surfactant. Preferred hydrophobic monoalkyl alcohols include, but are not limited to, 1-octanol, 1-nonanol, 1-decanol, 1-undecanol, 1-dodecanol, 1-tridecanol, 1-tetradecanol, 1-pentadecanol, 1-hexadecanol, 1-heptadecanol, 1-octadecanol, and 3,7,11,15-tetramethyl-2-hexadecen-1-ol (phytol). Long-chain alcohols with fewer than seven carbon atoms and more than 18 carbon atoms are not preferred. The antimicrobial composition can be free of short chain monohydric alcohols, such as methanol, ethanol, propanol, butanol, pentanol, hexanol, heptanol, isomers thereof, or any combination thereof. More preferred monoalkyl or monoalkylene alcohols are 1-dodecanol, 1-tridecanol, and 3,7,11,15-tetramethyl-2-hexadecen-1-ol (phytol).

Examples of suitable tonicity adjusting agents include but are not limited to: sodium chloride and potassium chloride, glycerin, propylene glycol, mannitol and sorbitol. These agents are typically used individually in amounts ranging from about 0.01 to 2.5% (w/v) or from about 0.05 to about 1.5% (w/v). In some embodiments, the tonicity agent will be employed in an amount to provide a final osmotic value of from 10 to 320 mOsm/kg, or from about 200 to about 300 mOsm/kg, or from about 260 to about 290 mOsm/Kg. In some embodiments, the tonicity is adjusted using sodium chloride.

In some embodiments, the pH of the antimicrobial solution is adjusted to between 4.5 to 7.0, or pH 5.0 to pH 6.5, or pH 5.5 to 6.0. The role of wound bed pH is of fundamental importance during healing. For instance, prolonged acidification of the wound bed has been shown to increase the healing rate in chronic venous leg ulcers (Wilson I. A. I., Henry M., Quill R. D., and Byrne P. J., VASA 1979, vol. 8, pages 339-342). The principal explanation for the mechanism of interaction between the acidic wound bed and the healing process is related to the potential to increase tissue oxygen availability through oxygen dissociation and to reduce the histotoxicity of bacterial end products, thus stimulating the injury's healing process.

Suitable buffers to adjust pH can include sodium citrate, potassium citrate, citric acid, sodium dihydrogen phosphate, disodium monophosphate, boric acid, sodium borate, tartrate, phthalate, succinate, acetate, propionate, maleate salts, tris(hydroxymethyl)aminomethane, amino alcohol buffers, and Good buffers (such as ACES, PIPES, and MOPOSO), and mixtures thereof. One or more buffers can be added to solutions of the present invention in amounts ranging between approximately 0.01 to 2.0 weight percent by volume, or from approximately 0.05 to 0.5 weight percent by volume.

Additionally, the pH of the antimicrobial composition can be adjusted by the combination of ethylenediaminetetracetic acid disodium and trisodium salt chelating agents.

The antimicrobial composition may be delivered in different forms, such as for example but not limited to a liquid, cream, foam, lotion, gel, powder, or aerosol. The antimicrobial composition can also be imbibed on swabs, cloth, sponges, foams, dressing materials and non-woven and paper products, such as paper towels and wipes. Topical formulations of the subject composition may additionally comprise organic solvents, emulsifiers, gelling agents, moisturizers, stabilizers, time release agents, dyes, perfumes, and like components commonly employed in formulations for biological hard tissue treatment.

In some embodiments, the antimicrobial composition may also be added to implanted devices in a hydrated or dried form to provide a coating that can be inserted into a body in order to prevent infection or biofilm attachment to the device.

Alternatively, the antimicrobial composition may be added to a solid or porous support and dried, such as a polymeric foam, and then applied directly to the biological hard tissue. In this case the polymeric foam may also absorb exudate, creating a hydrated environment for controlled release of the antimicrobial agents on the tissue surface.

### EXAMPLES

The following examples serve to illustrate the invention without limiting it thereby. It will be understood that variations and modifications can be made without departing from the spirit and scope of the invention.

The materials used in these examples include:

Antimicrobial compositions: The antimicrobial compositions used in these examples have previously been tested and found to be effective against the following microorganisms: Staphylococcus aureus, Escherichia coli, Pseudomonas aeruginosa, Staphylococcus epidermidis, Aspergillus brasiliensis, Methicillin Resistant Staphylococcus aureus (MRSA), Vancomycin Resistant Enterococcus faecalis (VREF), Serratia marcescens, Enterococcus faecalis, Proteus mirabilis, Trichosporin asahii, Clostridium difficile, Bacillus cereus, Acinetobacter baumannii, Candida auris, SARS-COV-2, Methicillin Resistant Staphylococcus epidermidis (MRSE), Cutibacterium acnes, Ralstonia pickettii, Enterobacter cloacae, Salmonella enterica, Candida albicans, Mucor circinelloides, Trichophyton rubrum.

BIASURGER Surgical Solution, Sanara Medtech, lot L7722N01, Ingredients: purified water, Poloxamer 407, sodium chloride, ethylhexylglycerin and octane-1,2-diol (0.4 wt % vicinal diols), hypromellose, PHMB (0.1 wt %), EDTAs (0.1 wt %).

BIAKÖS™ Antimicrobial Skin & Wound Cleanser, lot 17462, Ingredients: purified water, Poloxamer 407, sodium chloride, ethylhexylglycerin and octane-1,2-diol (0.4 wt % vicinal diols), hypromellose, PHMB (0.1 wt %), EDTAs (0.1 wt %).

BIAKÖS™ Antimicrobial Wound Gel, lot 18464, purified water, Poloxamer 407, sodium chloride, ethylhexylglycerin and octane-1,2-diol (0.4 wt % vicinal diols), PHMB (0.1 wt %), EDTAs (0.1 wt %).

### Biological Hard Tissue Samples:

Viable cancellous bone cores from human femoral heads, McGovern Medical School at UTHealth Houston (Bellaire TX)
Chicken bone (cleaned and air dried)
Human tooth (primary tooth from 6-year old)
Human hair (adult female, non-colored and colored portions)
Human nails (adult female, no nail polish)
Legume, dried uncooked pinto beans
Snake skin, shed garter snake skin at least 6 months old

### Tissue Culture Media:

Dulbecco's Modified Eagle Medium (DMEM) supplemented with 10% fetal bovine serum (FBS).

### Microbes:

Staphylococcus aureus ATCC 12600 (ATCC, Manassas, VA).

Example 1: Utilizing viable human bone explants, the bactericidal effects of the BIASURGE Surgical Solution were evaluated as well as bone tissue viability post treatment. This model employs bone explants fabricated from viable cancellous bone cores from human femoral heads resected during primary total hip arthroplasty. While the use of standard in vitro cell culture and microbial assays has place in the basic scientific research, use of more complex and relevant models, such as the ex vivo bone model described here, provides a more comprehensive picture of the effects of irrigation solutions on bone.

The effects of the antimicrobial treatment on the viability of the bone explants were evaluated by recovering surviving microorganisms from the bone explants at selected time points, followed by serial dilution and plating for enumeration. Microbial viability was evaluated immediately post treatment (acute) and 24 h post treatment (extended). Viability of the cells within the explants was determined using a resazurin assay which measures metabolic activity of the cells. The readout timepoints for this assay were 1 day and 13 days.

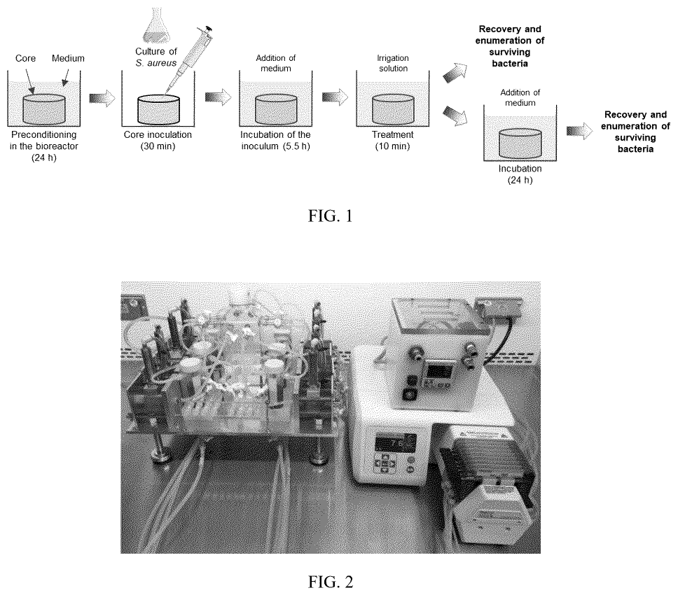

The cores were vortexed four times before inoculation to remove bone marrow and any prior antibiotic treatment. In the case of microbicidal effectiveness assessment, the cores (approximately ½″ in diameter) were placed in individual wells of a 6-well plate, inoculated with on average 1.4×10⁸ CFUs of S. aureus ATCC 12600, and incubated at 37° C. for 30 minutes. Dulbecco's Modified Eagle Medium (DMEM) was added to fully cover the cores and the samples were incubated an additional 5.5 hours at 37° C. Two core samples were then soaked in irrigation fluid and two core samples were soaked in saline for 10 minutes. Surviving bacteria were recovered and enumerated from one irrigation-soaked core and one saline-soaked core as soon as possible following the 10-minute soak. The remaining cores were incubated for an additional 24 hours and surviving bacteria recovered and enumerated at the 24 hour timepoint. A schematic of this process can be found in FIG. 1.

In the case of human cell/tissue viability assessment, after vortex wash, the cores were placed in a bioreactor, FIG. 2, and incubated for up to 13 days. Unlike static cultures, bioreactor systems maintain a controlled environment within which low molecular weight metabolites, waste products, and other macro-molecules can be transported while providing mechanical and chemical stimuli mimicking physiological conditions. The bone bioreactor system is comprised of 8 independent, autoclavable specimen chambers in which live bone cores are continuously loaded using programmable pneumatic actuators. As the response of bone cells to applied stress varies with frequency, the system allows for programmable cyclic loading coupled with intermittent or extended recovery periods. The media circulation control system provides a tunable fluid flow rate and allows for gas exchange, media sampling, and media replenishment through separate hypodermic injection ports to avoid cross contamination. An image of the system is shown in FIG. 2. The entire unit is housed within a water-jacketed incubator that maintains a 5% CO₂ environment at 37° C. The cores were first cultured for 1 day in static conditions, followed by 10-min treatment in the irrigation solution or saline (control) before placing the cores in the bioreactor, in contact with coupons of common ingrowth coatings of matching diameter to distribute loading across the surface. The system was set to circulate media and deliver a sinusoidal load across the bone-implant interface with recovery periods between each cycle. Two time points were evaluated here, 1 day (acute) and 13 days (extended) where the cores were washed and a resazurin assay was used to determine total metabolic activity of the cells within the explant. Metabolic activity of the treated explants was compared to the metabolic activity of explants exposed to a saline control (set to 100%).

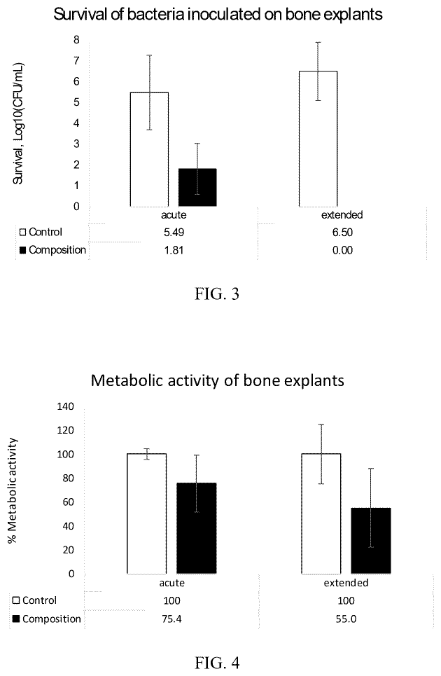

After 10 minutes of soaking in antimicrobial composition, high biocidal activity was evident with a 1.81 log survival of S. aureus compared to a 5.49 log survival with saline. After 24 hours of further incubation, there were no surviving bacteria with the antimicrobial composition treatment (10 minutes at time zero). However, the saline control exhibited an increase in viable microbial load with total number of bacteria about 6.50 logs. This data is shown in FIG. 3.

The metabolic activity of the bone cores exposed to a 10-minute soaking in the antimicrobial composition was 75.4% which demonstrated remarkable viability compared to other antimicrobial irrigation solutions (Lineaweaver, 1985). After 24 hours of further incubation, metabolic activity remains within 70-130% of the control (saline), which is deemed non-toxic as per ISO 10993-5 for 2D cell culture studies. In contrast, Lineweaver tested antimicrobial solutions of (i) 1% povidone-iodine, (ii) 0.25% acetic acid, (iii) 3% hydrogen peroxide, and (iv) 0.5% sodium hypochlorite, and found that each of them have cytotoxicity to human cells that exceed their bactericidal potency.

Example 2: Samples of human tooth, human hair, human nails, dried beans, snake skin, and chicken bone were submerged for 10 min in BIAKÖS antimicrobial cleanser solution. The samples of the hard tissue were placed in 50 mL centrifuge tubes followed by addition of approximately 4 mL of the antimicrobial solution (about 5 sprays), except chicken bone to which 25 mL of antimicrobial solution was added. The samples were incubated at room temperature (˜ 22° C.) for 10 min after which the solution was decanted, and the material evaluated for a variety of characteristics via visual observation. The results are summarized in Table 1 below.

TABLE 1
List of hard tissues evaluated and observations prior to/post treatment

| | | Observations post |
|---|---|---|
| Tissue | Observations prior to treatment | treatment |
| Human | Yellowish-white color | Same |
| tooth | Smooth surface | Same |
| | No observable surface damage | Same |
| | Readily wettable | Same |
| | Not sticky or tacky | Same |
| Hair | Smooth surface with a few (3-4) signs of | Same |
| | breakage in the field view of a hand-held | |
| | microscope (60x magnification) | |
| | Brown to blond color of strands (non-colored | Same |
| | and colored, respectively) | Same |
| | Not sticky, tacky, or slippery | |
| Nails | Off white color | Same |
| | Smooth surface | Same |
| | Flexible (bended at least 4 times without | Material sank |
| | breaking) | immediately |
| | When added water, the material first floated | |
| | before sinking to the bottom of the vessel | Same |
| | Not sticky, tacky, or slippery | |
| Dry | Light beige color to light brown with brown | Same |
| beans | freckles | More wrinkling of the |
| | Some wrinkling of the skin, otherwise smooth | skin, skin breakage in |
| | surface | some areas |
| | Not sticky, tacky, or slippery | Same |
| Snake | Two distinct sides to the material with different | Same |
| skin | flexibility and wettability | |
| | Wrapping paper texture | Material is more wet, |
| | | specifically top portion |
| | Not sticky, tack, or slippery | Same |
| | Bottom side curls up when exposed to water, | Same |
| | top side absorbs the water readily | |
| Chicken | Smooth texture except for one side which | Same |
| bone | appeared rougher | Same |
| | Off-white, grayish color | Same |
| | Not sticky, tacky, or slippery | |

There were no observable differences between the materials before vs after being submerged in the antimicrobial solution, with three exceptions, all of which relate to the water absorption properties of the materials. The exceptions were as follows: 1) nails that were exposed to the irrigation solution sank immediately when placed in a cup of water—this is expected considering that the material absorbs water when submerged in the solution, 2) dry beans that were submerged in the solution behaved similarly to beans submerged in water, and 3) snake skin post submersion stayed wet, in particular the top portion of the skin.

Example 3: A variety of antimicrobial composition form factors were applied onto samples of human nails using BIAKÖS antimicrobial cleanser solution or BIAKÖS antimicrobial wound gel. The following methods of application were used: liquid formulation, gel formulation, liquid formulation on a gauze pad, gel formulation on a gauze pad, liquid formulation on a cotton ball, gel formulation on a cotton ball, liquid formulation on a band-aid, gel formulation on a band-aid, dried liquid formulation applied as a tacky powder, dried gel formulation applied as paste. The sample tissues were covered with varying amounts of the form factors and within 2-3 min the form factors were removed, and the sample tissues wiped and evaluated visually.

All of the form factors described here allowed for complete coverage of the material and no injurious effects were observed. A summary of the observations can be found in Table 2.

TABLE 2
List of form factors applied to hard tissue
and observations prior to/post application.

| | Observations prior to | Observations post |
|---|---|---|
| Item | application | application |
| Liquid formulation | Off white color | No differences |
| Gel formulation | Smooth surface | No differences |
| Liquid formulation on gauze pad | Flexible (bended at least 4 | No differences |
| Gel formulation on gauze pad | times without breaking) | No differences |
| Liquid formulation on cotton | Not sticky, tacky, or | No differences |
| ball | slippery | |
| Gel formulation on cotton ball | | No differences |
| Liquid formulation on band-aid | | No differences |
| Gel formulation on band-aid | | No differences |
| Dried liquid formulation - tacky | | No differences |
| powder | | |
| Fried gel formulation - paste | No differences | |

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

What is claimed is:

1. A method of treating a biological hard tissue which comprises:
applying an antimicrobial composition to a biological hard tissue, wherein said antimicrobial composition comprises:
at least one polymeric biguanide;
a vicinal diol component comprising at least one monoalkyl glycol and at least one monoalkyl glycerol, wherein a weight ratio of said at least one polymeric biguanide and said vicinal diol component ranges from 1:0.05 to 1:500, and
a chelating agent, wherein a weight ratio of said at least one polymeric biguanide and said chelating agent ranges from 1:0.01 to 1:100.

2. The method of claim 1, wherein said antimicrobial composition is biocompatible with biological hard tissue.

3. The method of claim 1, wherein said application step occurs during a surgical procedure.

4. The method of claim 1, wherein said application step occurs during a surgical procedure where the biological hard tissue is accessed through an incision.

5. The method of claim 3, wherein the biological hard tissue is bone.

6. The method of claim 1, wherein the application step occurs via at least one of soaking, irrigation, gel coating, or powder coating of the biological hard tissue, or a surgical dressing or medical device to be contacted with biological hard tissue.

7. The method of claim 1, wherein the biological hard tissue is selected from bone, cementum, dentin, enamel, keratin, or sclerenchyma.

8. The method of claim 1, wherein the biological hard tissue is at least one of cementum, dentin, or enamel.

9. The method of claim 1, wherein the at least one polymeric biguanide is present in an amount of at least 0.05 weight %, wherein the percentage is based on the weight of the antimicrobial composition.

10. The method of claim 9, wherein the chelating agent is present at a concentration of from 0.01 weight % to 1 weight %, wherein the percentage is based on the weight of the antimicrobial composition.

11. The method of claim 1, wherein the chelating agent is present at a concentration of from 0.01 weight % to 1 weight %, wherein the percentage is based on the weight of the antimicrobial composition.

12. The method of claim 1, wherein said vicinal diol component is present in an amount ranging from 0.05 to 6.0 weight %, based on the total weight of the antimicrobial composition.

13. The method of claim 1, wherein the application step occurs via at least one of soaking, irrigation, gel coating, or powder deposition.

14. The method of claim 1, wherein the biological hard tissue is keratin.

15. The method of claim 1, wherein the application step occurs indirectly.

16. The method of claim 1, wherein the biological hard tissue is sclerenchyma.

17. The method of claim 1, wherein:
said at least one polymeric biguanide is present in an amount ranging from 0.05 to 1.5 weight %,
said vicinal diol component is present in an amount ranging from 0.05 to 6.0 weight %, based on the total weight of the antimicrobial composition, or
both.

18. The method of claim 1, wherein said vicinal diol component comprises at least one of the following:
(i) a monoalkyl glycol having the following structure: 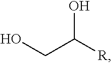
wherein R═C₃-C₁₈, branched or unbranched alkyl group or alkylene group; and
(ii) a monoalkyl glycerol having the following structure: 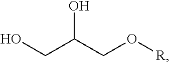
wherein R═C₃-C₁₈ branched or unbranched alkyl group or alkylene group.

19. The method of claim 1, wherein said polymeric biguanide comprises poly(hexamethylene biguanide) and its salts.

20. The method of claim 1, wherein the antimicrobial composition further comprises a bis(biguanide) at a concentration of from 10 ppm to 350 ppm.
